(19) United States
(12) Patent Application Publication
(10) Pub. No.: US 2009/0162943 A1
(43) Pub. Date: Jun. 25, 2009

(54) Reagents and Methods for the Detection of Transmissible Spongiform Encephalopathy

(75) Inventors: Ian McConnell, Cambridge (GB); Geoffrey John Russell Barnard, Cambridge (GB); John Coward, Cambridge (GB)

Correspondence Address: CLARK & ELBING LLP, 101 FEDERAL STREET, BOSTON, MA, 02110, US

(73) Assignee: .RevelationBio Limited, Boumemouth Dorset (GB)

(21) Appl. No.: 11/992,160

(22) PCT Filed: Sep. 20, 2006
(86) PCT No.: PCT/GB2006/003494

§ 371 Date: Mar. 17, 2008

(30) Foreign Application Priority Data
Sep. 20, 2005 (GB) 0519198.6

(51) Int. Cl. G01N 33/566

(52) U.S. Cl. 436/501

(57) ABSTRACT

This invention relates to the detection of transmissible spongiform encephalopathy (TSE) in samples using agents, such as antibodies, that bind to the extreme N-terminal region of mature full-length PrP. These allow the development of sensitive immunoassays for PrP^{d} for use in the detection of TSE without the need for proteolysis.

This invention relates to methods and materials for the detection of transmissible spongiform encephalopathy (TSE).

The conversion of the cellular prion protein (normally designated PrP^{C}) to a disease-associated isoform (hereinafter designated PrP^{d}) is the key process in the pathogenesis of TSE (Prusiner 1998). Consequently, the specific detection of PrP^{d} forms the basis for the biochemical diagnosis of TSE, which includes bovine spongiform encephalopathy (BSE) in cattle, scrapie in sheep, hamsters and mice, chronic wasting disease in deer and elk and Creutzfeld-Jacob Disease (CJD) in humans (Kascsak, Fersko et al. 1997; Kubler, Oesch et al. 2003).

Known techniques for the specific detection of PrP^{d} generally involve the differential proteolysis of PrP^{C} using enzymes such as proteinase K (PK) or trypsin, followed by the detection of a protease-resistant core of PrP (normally designated PrP^{res}) by Western blotting (Madec, Groschup et al. 1998; Schaller, Fatzer et al. 1999; Madec, Belli et al. 2000; Oesch, Doherr et al. 2000; Cooley, Clark et al. 2001; Stack, Chaplin et al. 2002) or other immunochemical means (Moynagh and Schimmel 1999; Grassi, Comoy et al. 2001; Biffiger, Zwald et al. 2002; Safar, Scott et al. 2002).

However, careful optimisation is required to identify the exact conditions of proteolysis which ensure that the detection of PrP^{res} is a reflection of the presence of disease-associated PrP (i.e. PrP^{d}). Moreover, conditions that have been optimised for the detection of PrP^{res} in one type of tissue (e.g. brain stem) from an animal in the terminal stages of the disease may be inappropriate for the detection of PrP^{res} in primary and secondary lymphoid tissue or other body fluids (e.g. blood), which is necessary for the detection of pre-clinical disease. In these situations, optimum conditions for proteolysis may need to be constantly readjusted and difficult to control. Another problem with proteolysis is the constraints arising from high-throughput automation, where inadequate control of proteolysis may give rise to false positives and/or false negatives.

Various monoclonal antibodies (Mabs) that are specific for PrP^{d} have been reported (Korth, Stierli et al. 1997; Curin Serbec, Bresjanac et al. 2003; Paramithiotis, Pinard et al. 2003). All these Mabs have relative low affinity and recognise conformational epitopes. This means that PrP^{d} denaturation precludes the binding of these antibodies. Without denaturation, the vast majority of PrP^{d} is ‘locked’ in its aggregated state. Consequently, the use of these antibodies may be somewhat limited and the sensitivity of detection greatly reduced.

The present inventors have unexpectedly recognized that agents that bind to the extreme N-terminal region of mature full-length PrP are useful in methods for the detection of PrP^{d}. that are operationally specific. This allows the development of sensitive immunoassays for PrP^{d} without the need for proteolysis.

One aspect of the invention provides a specific binding member which binds to an amino acid sequence selected from the group consisting of SEQ ID NO: 1, SEQ ID NO: 2 and SEQ ID NO: 3.

SEQ ID NO: 1, SEQ ID NO: 2 and SEQ ID NO: 3 represent the extreme N-terminus of full-length human, ruminant and murine PrPs, respectively.

A specific binding member as described herein may bind to human, ruminant (including ovine, bovine and cervine) and/or murine PrP^{d}.

A specific binding member which binds to an amino acid sequence selected from the group consisting of SEQ ID NO: 1, SEQ ID NO: 2 and SEQ ID NO: 3 may bind specifically to a linear epitope within one or more, two or more or all three of these amino acid sequences. For example, a specific binding member may bind to an epitope consisting of 6 or more, 7 or more, 8 or more, 9 or more, 10 or more or 11 or more contiguous amino acids from the amino acid sequence of SEQ ID NO: 1, SEQ ID NO: 2 and/or SEQ ID NO: 3.

In some embodiments, the specific binding member may bind to the amino acid sequence KKRPKPGGGWNT or KKRPKPGGWNT or may bind specifically to an epitope within the amino acid sequence KKRPKPGGGWNT and/or KKRPKPGGWNT. In some preferred embodiments, the specific binding member may bind to the linear epitope KKRPKPG or to the linear epitope PGGGWNT.

A specific binding member as described herein may bind to PrP^{d} extracted from biological material by procedures described herein. Preferably, the specific binding member binds specifically to PrP^{d} i.e. the antibody binds to PrP^{d} but shows little or no binding to PrP^{c} from the same source, for example PrP^{c} extracted from biological material as described herein.

Preferably, a specific binding member binds to both denatured PrP^{d} and native PrP^{d}, but shows little or no binding to PrP^{c} in either its denatured or native conformations. Recombinant PrP does not undergo the post-translational processes which may remove or mask the N terminal of PrP^{c}. Accordingly, a specific binding member as described herein may also bind to recombinant PrP.

A specific binding member may bind to the extreme N terminal region of the PrP sequence, for example, the extreme N terminal region of the human, bovine, ovine or murine PrP sequence. For example, the specific binding member may bind to the region between the N terminal and the first tyrosine residue in the PrP sequence (e.g. Tyr38 in human PrP and Tyr41 in bovine PrP) i.e. the region consisting of the N terminal 38 or 40 amino acids of PrP.

A specific binding member which binds to a linear epitope within SEQ ID NO: 1, which is the extreme N terminal region of full-length human PrP including the signal peptide, in particular a specific binding member which binds the amino acid sequence KKRPKPGGWNT, may be particularly useful in binding to human PrP^{d}. A specific binding member which binds specifically to a linear epitope within SEQ ID NO: 2, which is the N terminal of full-length bovine PrP including the signal peptide, in particular the amino acid sequence KKRPKPGGGWNT, may be particularly useful in binding to bovine, ovine or cervine PrP^{d}. A specific binding member which binds to a linear epitope within SEQ ID NO: 3, which is the extreme N terminal region of full-length murine PrP including the signal peptide, in particular a specific binding member which binds the amino acid sequence KKRPKPGGWNT, may be particularly useful in binding to murine PrP^{d}.

Antibodies according to the invention include the YWH1, YWH2 and YWH3 antibodies described herein.

YWH1 is produced by the hybridoma designated ‘YWH1’ which was deposited on 1 Sep. 2005 at ECCAC, Porton Down, Wiltshire, UK with the Provisional Accession Number 05091301 by John Coward. A YWH1 antibody as described herein may have the V_{H}CDR1, V_{H}CDR2 and V_{H}CDR3 sequences and V_{L}CDR1, V_{L}CDR2 and V_{L}CDR3 sequences of the antibody produced by the deposited hybridoma, more preferably, the antibody has the V^{L} and V_{H} regions of the antibody produced by the deposited hybridoma. In some embodiments, a YWH1 antibody may be produced by the deposited hybridoma or may be identical to the antibody produced by the deposited hybridoma. In other embodiments, a YWH1 antibody as described herein may compete for binding to PrP^{d} with the antibody produced by the deposited hybridoma.

A binding member which specifically binds to a target epitope (i.e. a specific binding member) may show significant binding to the target epitope (e.g. a target in the extreme N terminal region of PrP) when the epitope is present and accessible in a polypeptide, (e.g. PrP^{d}), and may show little or no binding to peptides or polypeptides in which the target epitope is absent or inaccessible to specific binding member (e.g. PrP^{c}). In particular, a specific binding member may show no significant binding to other proteins present in the sample, in particular other proteins present in mammalian brain, skin, tonsil or lymphoid tissue samples or samples of biological fluid such as blood, saliva or urine. In addition, an antibody which specifically binds within the extreme N terminal region of PrP^{d} may show no significant binding to PrP^{d} outside this region.

Generally, a specific binding member which specifically binds to a target epitope may have a binding affinity which is greater than about 10⁵ moles/litre (e.g., 10⁶, 10⁷, 10⁸, 10⁹, 10¹⁰, 10¹¹, and 10¹² or more moles/litre).

A specific binding member is member of a pair of molecules that have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organisation of the other member of the pair of molecules. Thus, the members of the pair have the property of binding specifically to each other. Examples of types of specific binding pairs are antigen-antibody, biotin-avidin, hormone-hormone receptor, receptor-ligand, enzyme-substrate. A specific binding member as described herein may comprise immunoglobulin or other amino acid sequences, e.g. forming a peptide or polypeptide, such as a folded domain, or to impart to the molecule another functional characteristic in addition to ability to bind antigen.

Preferred specific binding members include antibody molecules. An antibody molecule is an immunoglobulin that may be natural or partly or wholly synthetically produced. The term includes any polypeptide or protein comprising an antibody-binding domain, including antibodies and antibody fragments. Antibody fragments that comprise an antigen-binding domain include Fab, scFv, Fv, dAb, Fd, and diabodies.

In some preferred embodiments, a specific binding member described herein may be a monoclonal or polyclonal antibody.

A method of producing an antibody as described herein may comprise:
administering an immunogen comprising a sequence selected from the group consisting of SEQ ID NOS: 1 to 3 and fragments thereof to an animal, and;
isolating from said animal an antibody which binds to said peptide sequence.

A fragment of an amino acid sequence may comprise 6 or more, 7 or more, 8 or more, 9 or more, 10 or more or 11 or more contiguous amino acids from the amino acid sequence of SEQ ID NO: 1, SEQ ID NO: 2 or SEQ ID NO: 3. Suitable fragments include KKRPKPGGGWNT, KKRPKPGGWNT, KKRPKPG and PGGGWNT.

The immunogen may comprise a protein carrier, such as Keyhole Limpet Haemocyanin. Other suitable carriers are well known in the art.

In some embodiments, the immunogen may comprise a chimeric peptide consisting of amino acids 25-36 of the ovine or bovine PrP sequence and, for example amino acids 62-69 of the ovine or bovine PrP sequence or to a chimeric peptide consisting of amino acids 23-34 of the human PrP sequence and, for example, amino acids 59-66 of the human PrP sequence.

Antibodies may be obtained from immunised animals using any of a variety of techniques known in the art, and screened, preferably using binding of antibody to antigen of interest. For instance, Western blotting techniques, immunoassay or immunoprecipitation may be used (Armitage et al. (1992) Nature 357 80-82).

More preferably, an antibody molecule may be a monoclonal antibody. Methods of producing monoclonal antibodies are well known in the art (see, for example, Harlow et al Antibodies: A Laboratory Manual, Cold Spring Harbor. Laboratory (Cold Spring Harbor, N.Y., 1988) pp. 353-355) and are described in more detail below. For example, antibody-producing cells may be isolated from an immunised mammal and fused with immortalised cells to produce a population of antibody-producing hybridoma cells, which can then be screened to identify a hybridoma cell that produces an antibody which displays optimal binding characteristics.

In some embodiments, a hybridoma may be produced by a method comprising;
immunising a non-human mammal with an immunogen comprising a sequence selected from the group consisting of SEQ ID NOS: 1 to 3 and fragments thereof,
producing one or more fusions of antibody producing cells from said mammal and immortalised cells to provide a population of hybridoma cells, and;
screening said population to identify a hybridoma cell which produces an antibody which binds the sequence selected from the group consisting of SEQ ID NOS: 1 to 3 and fragments thereof.

The population of hybridoma cells is preferably screened by testing the binding of antibodies produced by cells of the population to one or more peptide sequences selected from the group consisting of SEQ ID NOS: 1 to 3 and fragments thereof, such as KKRPKPGGGWNT or KKRPKPGGWNT or peptide epitopes within one of these sequences. For instance, western blotting techniques, immunoassay or immunoprecipitation may be used.

Hybridoma cells identified as producing antibodies which bind to an epitope within the extreme N terminal sequence of PrP (e.g. amino acids 1-40 of bovine PrP or amino acids 1-38 of human PrP, preferably amino acids 25-36 of bovine PrP or amino acids 23-34 of human PrP) may be isolated and/or purified from the population.

Following isolation, the hybridoma may be expanded, maintained and/or cultured in a culture medium using methods which are well-known in the art. Antibodies produced by the hybridoma may be isolated from said culture medium. A method of producing an antibody as described herein may comprise;
culturing a hybridoma cell produced as described above in a culture medium; and,
isolating from the medium an antibody as described above, for example, an antibody which binds to one or more sequences selected from the group consisting of SEQ ID NOS: 1 to 3 and fragments thereof, such as KKRPKPGGGWNT or KKRPKPGGWNT.

Alternatively, a specific binding member may be produced using any of a plethora of known display technologies and combinatorial libraries (Rothe A et al. FASEB J. 2006; 20:1599-610). Initially, the immunoglobulin V-region was used as a scaffold for modification using recombinant techniques but recently many approaches have been adopted. Two examples include the involvement of the fibronectin type III domain, (Koide A et al. Journal of Molecular Biology 1998; 284:1141-51). Another example involves the use of a carbohydrate-binding module from a xylanase found in Rhodothermus marinus (Cicortas Gunnarsson L et al. Protein Eng. Des. Sel. 2004; 17:213-21). Alternative binding partners to PrP (e.g. nucleic acids, glycosaminoglycans etc.) may be discovered by other procedures known in the art.

In a preferred embodiment, a monoclonal antibody specific for an epitope within the N terminal sequence of PrP may be obtained from a recombinantly produced library of expressed immunoglobulin variable domains or other molecules comprising antibody antigen-binding domains, e.g. using lambda bacteriophage or filamentous bacteriophage which display functional immunoglobulin binding domains on their surfaces; for instance see WO92/01047. The library may be immunologically naive, that is constructed from sequences obtained from an organism which has not been immunised with a peptide comprising the epitope, or may be one constructed using sequences obtained from an organism which has been exposed to the antigen of interest. A method of raising an antibody as described herein may comprise:
contacting an immunogen comprising the peptide sequence selected from the group consisting of SEQ ID NOS: 1 to 3 and fragments thereof with a diverse population of antibody antigen-binding domains; and,
determining the binding of members of said population to said peptide.

In some embodiments, an immunogen may comprise a chimeric peptide consisting of amino acids 25-36 of the ovine or bovine PrP sequence, and, for example, amino acids 62-69 of the ovine or bovine PrP sequence, or a chimeric peptide consisting of amino acids 23-34 of the human PrP sequence and, for example, amino acids 59-66 of the human PrP sequence.

Antibody antigen-binding domains may be displayed on the surface of virus particles i.e. the diverse population may be a phage display library.

A method may comprise identifying an antibody antigen-binding domain within said population which binds to an amino acid sequence selected from the group consisting of SEQ ID NOS: 1 to 3 and fragments thereof, such as KKRPKPGGGWNT or KKRPKPGGWNT.

The virus particle which displays the identified antibody antigen-binding domain may be isolated and/or purified and the nucleic acid encoding the antibody antigen-binding domain obtained from said particle.

The nucleic acid encoding the antibody antigen-binding domain may be sequenced and/or expressed to produce the encoded antibody antigen-binding domain that binds to an epitope within the peptide sequence of SEQ ID NO: 1, SEQ ID NO: 2 and/or SEQ ID NO: 3.

An antibody antigen-binding domain produced as described above may be further tested using routine methodology to determine its specificity i.e. to determine whether it binds specifically to one or both of the peptide sequences.

In some embodiments, the binding properties of the antibody antigen-binding domain may be further optimised using standard antibody engineering techniques, including affinity maturation, for example by chain shuffling, and site-specific, random or combinatorial mutagenesis.

An antibody antigen-binding domain which is comprised in an antibody or antibody molecule, for example an scFv, Fab, Fv, dAb, Fd or diabody molecule, may be reformatted, for example into an IgG antibody, using standard techniques for subsequent use.

Another aspect of the invention provides a method of detecting a TSE in an individual comprising
contacting a sample obtained from the individual with an specific binding member as described above, for example an antibody molecule which binds specifically to an amino acid sequence selected from the group consisting of SEQ ID NOS: 1 to 3 and fragments thereof, such as KKRPKPGGGWNT or KKRPKPGGWNT, and;
determining the binding of said antibody to said sample.

Binding of the antibody to the sample is indicative of the presence of PrP^{d} in the sample, which is indicative that the individual has TSE.

The amount of binding may be indicative of the amount of PrP^{d} in the sample. This may be indicative of the extent or progression of the TSE in the individual.

Binding of the antibody may be determined relative to controls. Suitable controls may include tissue from a healthy individual (i.e. an individual not suffering from TSE).

Samples may be prepared, for example, from nervous tissue (e.g. brain tissue), from lymphoid tissue (e.g. primary or secondary lymphoid tissue) or from cells (e.g. white blood cells, fibroblasts) obtained from the individual. Samples may also be prepared from body fluids (e.g. blood, saliva or urine).

In some preferred embodiments, primary lymphoid tissue or secondary lymphoid tissue from the individual is used.

Tissues and fluids obtained from the individual may be treated to solubilise and extract insoluble protein aggregates and fragments to facilitate their detection in an immunoassay.

Suitable treatments may include treatment with a solubilising agent, such as a solvent or chaotropic agent, chemical treatment with salt solutions and physical treatments such as sonication.

A chaotropic agent is a reagent that denatures proteins and, in particular, may facilitate the extraction of protein aggregates and fragments. Suitable chaotropic agents are well known in the art and include guanidine hydrochloride (GdHCl) and urea. The chaotropic agent may be used at any concentration that produces a denaturant effect, for example, 8M GdHCl may be employed.

Suitable solvents are also well known in the art and include methanol. Suitable salt solutions include saturated ammonium sulphate.

Tissue obtained from the individual may be disrupted and/or homogenised before contact with the antibody. For example, the tissue may be homogenised in the presence of the chaotropic agent in the presence or absence of detergent, such as Tween-20™ or Triton X-100™.

Preferably the tissue or extract is not subjected to protease treatment (i.e. the tissue or extract is non-protease treated), prior to contact with said antibody.

In some embodiments, the individual is a non-human animal. The methods described herein may be used to detect scrapie in sheep and goats, experimental scrapie in hamsters and mice, chronic wasting disease in elk and deer (cervids) and/or BSE in cattle or other animals.

In other embodiments, the individual is a human and the methods described herein may be used to detect Creutzfeldt-Jakob disease (CJD), Kuru, Gerstmann-Straussler-Scheinker syndrome or familial fatal insomnia (FFI).

The individual may display no behavioural changes or clinical symptoms associated with TSE or, alternatively, the individual may display one or more behavioural changes or clinical symptoms associated with TSE.

Specific binding members which bind to sequence selected from the group consisting of SEQ ID NOS: 1 to 3 and fragments thereof, such as KKRPKPGGGWNT or KKRPKPGGWNT, and which may specifically bind PrP^{d}, are described above. In some embodiments, the specific binding member may comprise a detectable label or a tag which binds to a detectable label. Binding of the specific binding member to the sample may be determined by detecting the presence of the label. The level, amount or concentration of PrP^{d} in the sample may be determined from the amount of label detected.

Suitable labels are described in more detail below.

In other embodiments, binding of the specific binding member may be determined using a second specific binding member which binds to the first specific binding member and which comprises a detectable label.

Conveniently, a sandwich assay format may be employed. A method of detecting a TSE in an individual may comprise;
contacting a sample from said individual with a first specific binding member; and,
determining binding of said first specific binding member to a PrP^{d} molecule in said sample using a second specific binding member,
the presence of said binding being indicative of the presence of TSE in the individual, wherein one of said first or second specific binding members binds to the sequence selected from the group consisting of SEQ ID NOS: 1 to 3 and fragments thereof, such as KKRPKPGGGWNT or KKRPKPGGWNT and the other of said first or second specific binding members binds to both PrP^{c} and PrP^{d}.

For example, the first specific binding member may bind to the sequence KKRPKPGGGWNT or KKRPKPGGWNT and the second specific binding member may bind to PrP^{c} and PrP^{d} or the second specific binding member may bind to the sequence KKRPKPGGGWNT or KKRPKPGGWNT and the first specific binding member may bind to PrP^{c} and PrP^{d}.

Suitable specific binding members may include carbohydrates, glycosaminoglycans, nucleic acids, peptides and proteins including antibody molecules.

Specific binding members, such as antibodies, which bind to PrP^{c} and PrP^{d} are well-known in the art and may, for example, recognise epitopes common to both protein forms, such as the octapeptide repeat region spanning residues 51 to 98 of the PrP sequence.

One of said first and second specific binding members may be immobilised and the binding of the other specific binding member may be detected. Preferably, the first specific binding member is immobilised. A specific binding member may be immobilised, for example, by attachment to an insoluble support. The support may be in particulate or solid form and may include a plate, a test tube, beads, a ball, a filter or a membrane. A specific binding member may, for example, be fixed to an insoluble support that is suitable for use in affinity chromatography. Methods for fixing specific binding members to insoluble supports are known to those skilled in the art.

The non-immobilised specific binding member may comprise a detectable label. For example, the antibody may be labelled with a fluorophore, preferably a lanthanide such as Europium, a radioisotope, an enzyme or particle or a non-isotopic labeling reagent such as biotin or digoxigenin or any other detectable ‘Tag’ or label, which may for example be a unique small molecule, peptide or nucleic acid.

In some embodiments, the non-immobilised specific binding member may be detected using a third specific binding member which binds to the non-immobilised specific binding member. The third antibody may comprise a detectable label.

Linkage of detectable labels to a specific binding member may be direct or indirect, covalent, e.g. via a peptide bond, or non-covalent. Linkage via a peptide bond may be as a result of recombinant expression of a gene fusion encoding specific binding member (e.g. antibody) and label molecule. Linkage via a non-covalent bond may be a result of a binding between a biotinylated antibody and a streptavidin/avidin linked label molecule.

Labels include fluorochromes such as fluorescein, rhodamine, phycoerythrin, Europium and Texas Red, chromogenic dyes such as diaminobenzidine, macromolecular colloidal particles or particulate material such as latex beads that are coloured, magnetic or paramagnetic, metal sols (e.g. gold sol), dye sols, and biologically or chemically active agents that can directly or indirectly cause detectable signals to be visually observed, electronically detected or otherwise recorded, for example by FACS, ELISA, Western blot, TRFIA, immunohistochemistry, or immunoassay, including lateral flow assays such as immunochromatographic strips, flow-through assays, agglutination assays or solid-phase assays such as dipstick or dipstick comb assays. The preparation and use of suitably labelled molecules is well-known in the art (see for example EP0291194B1).

Biologically or chemically active agents include enzymes, which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed. Further examples include horseradish peroxidase and chemiluminescence.

In some preferred embodiments, the detectable label is a lanthanide element such as Europium (Eu), Samarium (Sm), Terbium (Tb) or Dysprosium (Dy). Because the fluorescence of lanthanides such as Europium decays over a much longer time period than natural fluorescence, these labels may be used in time resolved fluorometric immunoassays (TRFIA) to reduce background and increase sensitivity.

In other preferred embodiments, the antibodies may be configured in an immunoassay device, including a solid-phase device such as a dipstick, a flow-through device or a lateral flow device. Suitable devices are well known in the art (see for example GB 1589234, EP 0225054, EP 0183442, EP0291194 and EP 0186799).

Although methods of the invention may be performed in any convenient assay format, such as FACS, ELISA, Western blot, TRFIA, immunohistochemistry, or immunoassay, such as a lateral flow, flow-through, agglutination or solid-phase assay, in some especially preferred embodiments the methods described herein are performed in a TRFIA format, such as DELFIA™ (dissociation enhanced lanthanide fluorescence immunoassay).

DELFIA™ employs lanthanide labels in combination with an Enhancement solution to produce a fluorescent signal which is detected on a time resolved fluorimeter. DELFIA™ assays are well known in the art (see for example Allicotti et al J Immunoassay Immunochem. 2003; 24(4): 345-58; Butcher et al J Immunol Methods. 2003 Jan. 15; 272(1-2): 247-56).

A method may comprise contacting the detectable label with a fluorescent enhancer and detecting the fluorescence from said label.

The mode of determining binding is not a feature of the present invention and those skilled in the art are able to choose a suitable mode according to their preference and general knowledge.

Other aspects of the invention provide the use of a peptide consisting of a sequence selected from the group consisting of SEQ ID NOS: 1 to 3 and fragments thereof or a chimeric peptide consisting of amino acids 25-36 of the ovine or bovine PrP sequence and, for example, amino acids 62-69 of the ovine or bovine PrP sequence or a chimeric peptide consisting of amino acids 23-34 of the human PrP sequence and, for example, amino acids 59-66 of the human PrP sequence in the production of specific binding members such as antibody molecules for use in detecting TSE and the use of a specific binding member such as an antibody molecule which specifically binds to an amino acid sequence selected from the group consisting of SEQ ID NOS: 1 to 3 and fragments thereof, such as KKRPKPGGGWNT or KKRPKPGGWNT, in the detection of TSE.

A specific binding member such as antibody molecule as described herein may form part of a kit for detecting or diagnosing a TSE e.g. in a suitable container such as a vial in which the contents are protected from the external environment.

A kit for detecting TSE or assessing the progression of TSE in an individual may comprise:
one or more specific binding members such as antibody molecules which specifically bind to an amino acid sequence selected from the group consisting of SEQ ID NOS: 1 to 3 and fragments thereof, such as KKRPKPGGGWNT or KKRPKPGGWNT, as described above, and;
detection reagents for determining binding of said one or more specific binding members to a sample obtained from the individual.

The one or more specific binding members may be free in solution or may be immobilised on a solid support, such as a magnetic bead, tube, membrane or microplate well.

Detection reagents may comprise a second specific binding member which binds to both PrP^{c} and PrP^{d}. The PrP^{d} specific specific binding member or the second specific binding member may be labelled with a detectable label, for example a lanthanide such as Europium, a gold or other sol particle, coloured bead or a tag which binds a detectable label. Suitable tags include biotin. If the specific binding members are labelled with a tag, the detection reagents may further comprise a detectable label that binds to the tag.

The detection reagents may further comprise a substrate, for example a chromogenic, fluorescent or chemiluminescent substrate, which reacts with the label to produce a signal. Substrates may include enhancer solutions and inducers for use in TRFIA assays such as DELFIA®, including DELFIA® enhancement and wash solutions as described herein. The detection reagents may further comprise buffer solutions, wash solutions etc.

The kit may also comprise one or both of: apparatus for handling and/or storing tissue obtained from the individual; and, reagents for homogenising and/or extracting said sample from said tissue, for example chaotropic agents such as GdHCl with or without detergent.

The kit may also include instructions for use of the antibody molecule or other specific binding member, e.g. in a method of detecting a TSE disorder in a test sample, as described herein.

Various further aspects and embodiments of the present invention will be apparent to those skilled in the art in view of the present disclosure. All documents mentioned in this specification are incorporated herein by reference in their entirety.

The invention encompasses each and every combination and sub-combination of the features that are described above.

Certain aspects and embodiments of the invention will now be illustrated by way of example and with reference to the figures described above and tables described below.

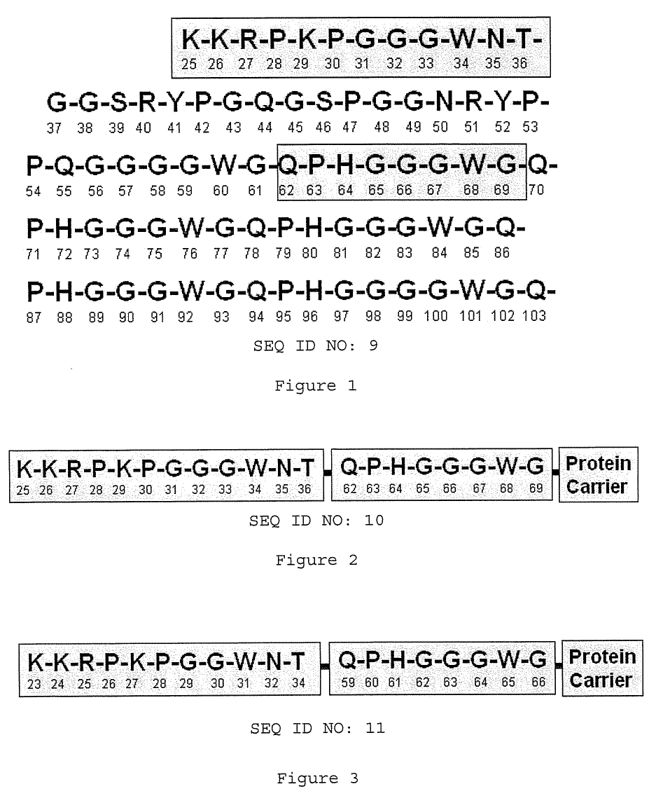

FIG. 1 shows the N-terminal sequence of bovine PrP identifying peptides selected for the construction of the immunogen

FIG. 2 shows the N-terminal bovine chimeric peptide used for the immunogen for the production of YWH1

FIG. 3 shows the N-terminal human chimeric peptide used as the antigen in the panning for YWH2 and YWH3.

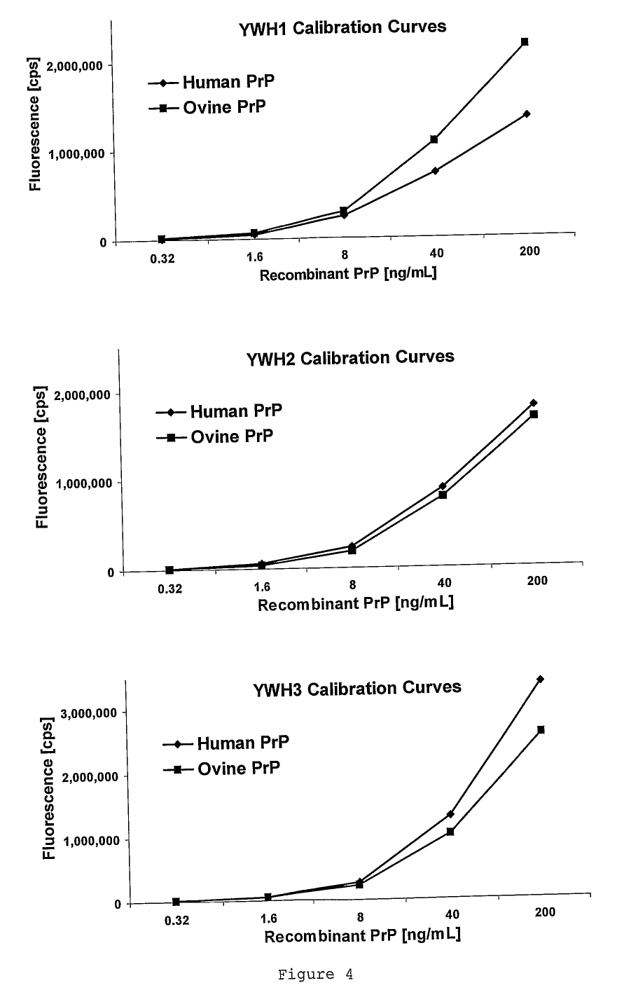

FIG. 4 shows calibration curves of ovine and human recombinant PrP for YWH1, YWH2 and YWH3 (linear Y-axis)

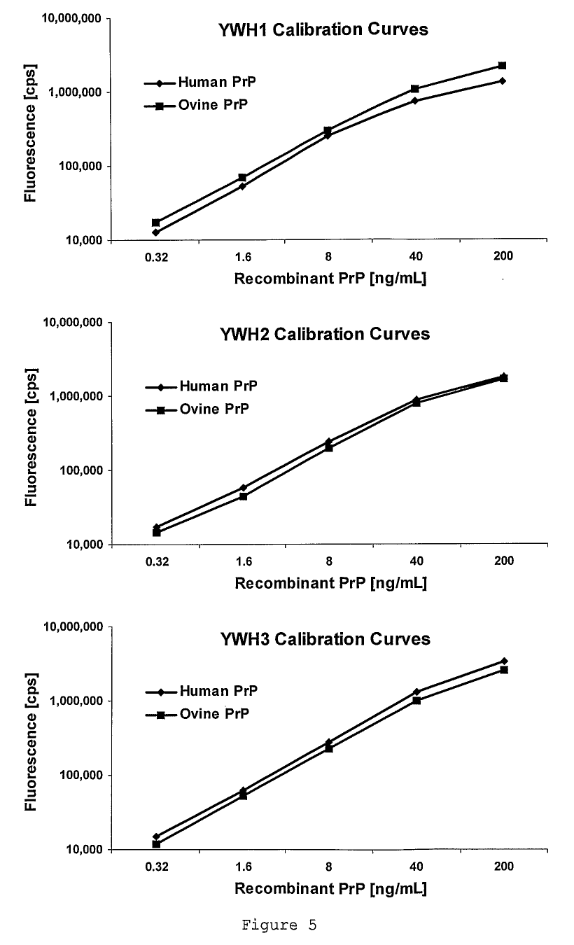

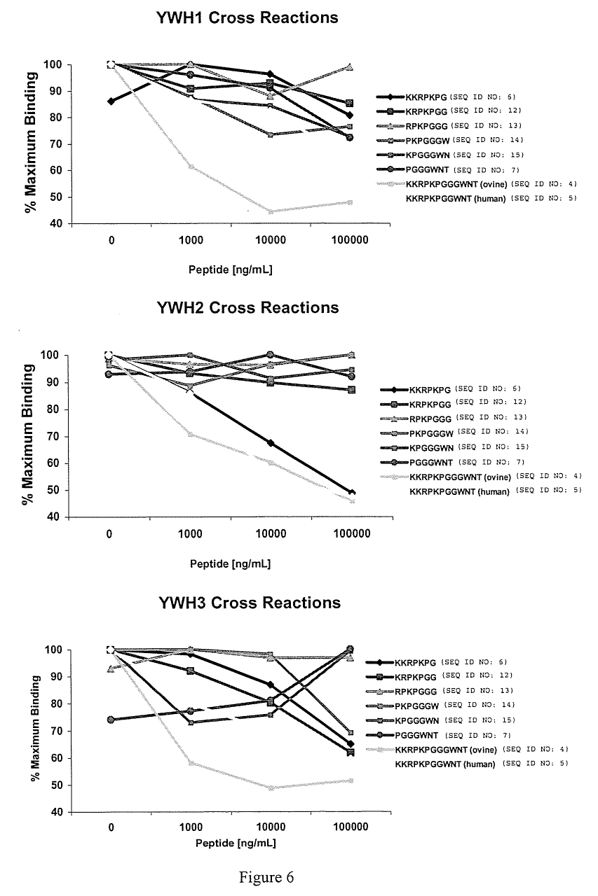

FIG. 5 shows calibration curves of ovine and human recombinant PrP for YWH1, YWH2 and YWH3 (logarithmic Y-axis)

FIG. 6 shows cross-reactions of YWH1, YWH2 and YWH3

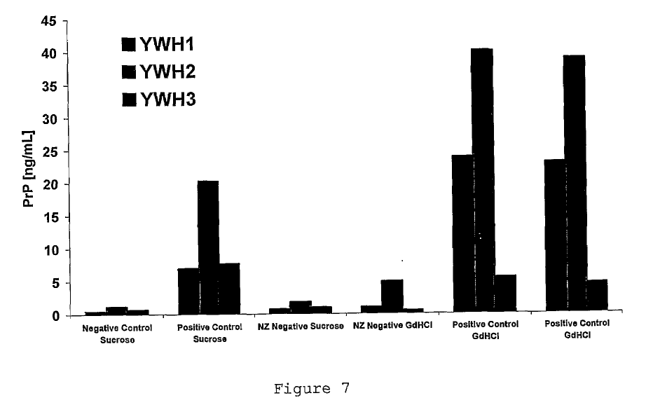

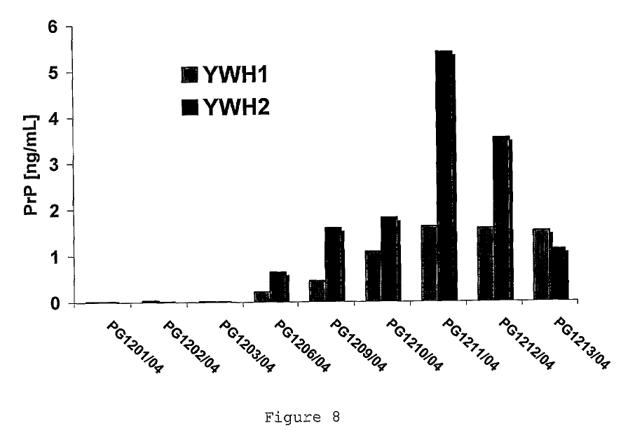

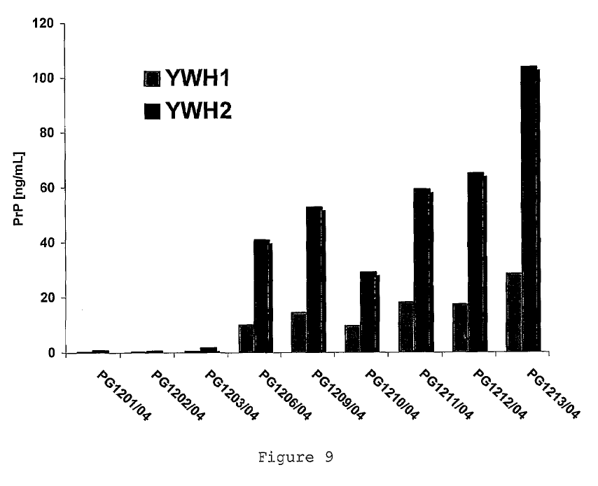

FIG. 7 shows measurement of disease-associated PrP using YWH series antibodies in scrapie positive and negative tissues following homogenisation in sucrose or GdHCl

FIG. 8 shows measurement of disease-associated PrP in histology-confirmed scrapie positive and negative ovine palatine tonsil. PG1201/04, PG1202/04 and PG 1203/04 are histology-confirmed scrapie negative tissues

FIG. 9 shows measurement of disease-associated PrP in histology-confirmed scrapie positive and negative rostral medulla.

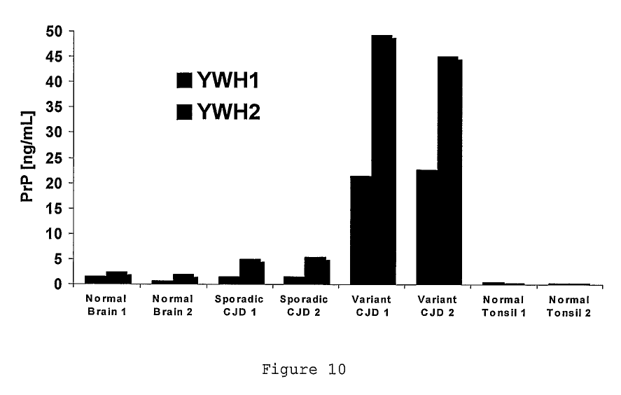

FIG. 10 shows measurement of disease-associated PrP in human brain and tonsil homogenate.

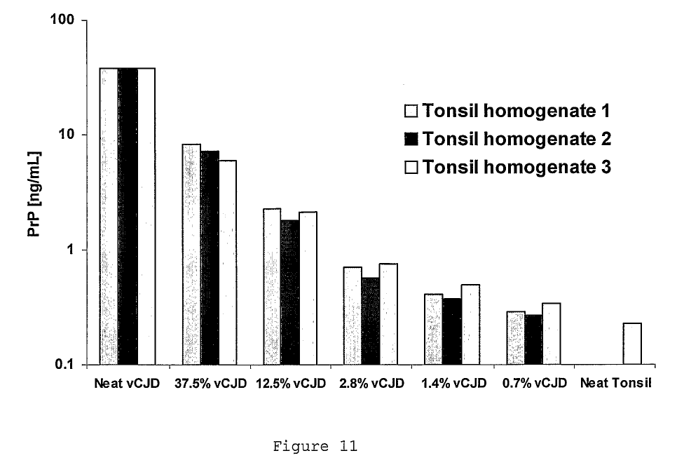

FIG. 11 shows measurement of disease-associated PrP in vCJD-spiked human tonsil homogenate.

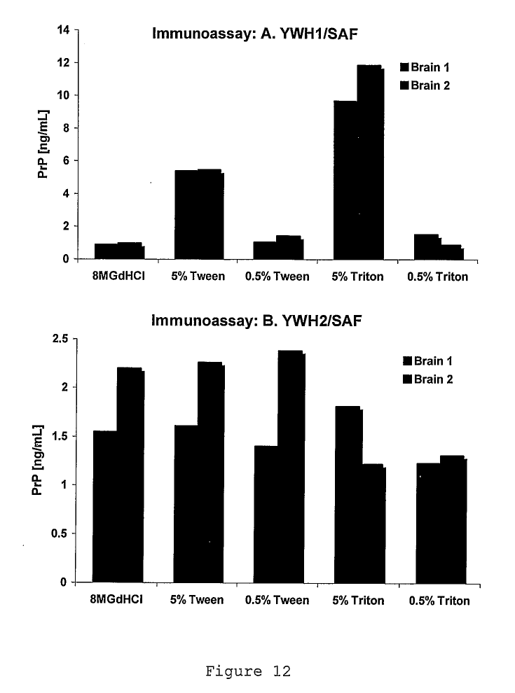

FIG. 12 shows the impact of involving detergent in the homogenisation of negative brain tissue with 8M GDHCl.

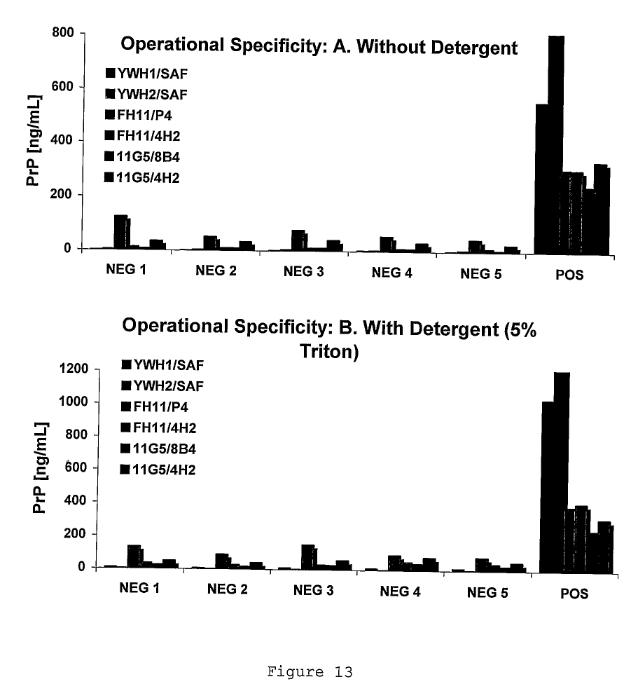

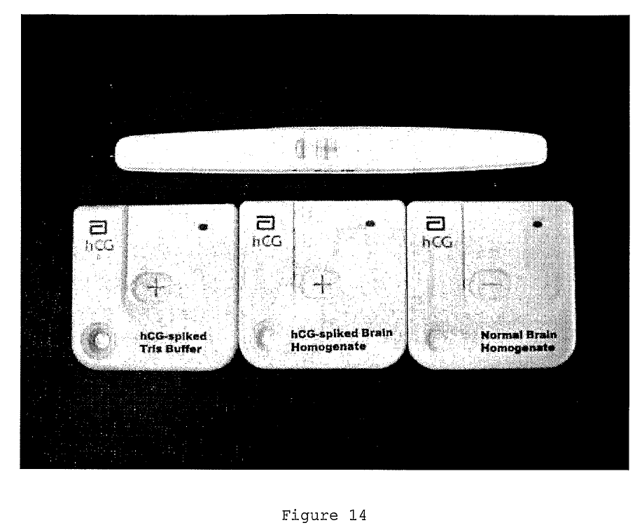

FIG. 13 shows the impact of involving alternative antibody combinations in the determination of disease-associated PrP in brain tissue homogenates with (FIG. 13B) and without detergent (FIG. 13A).

FIG. 14 demonstrates that homogenates prepared as described and spiked with human chorionic gonadotrophin do not interfere with the lateral flow of a dipstick pregnancy test.

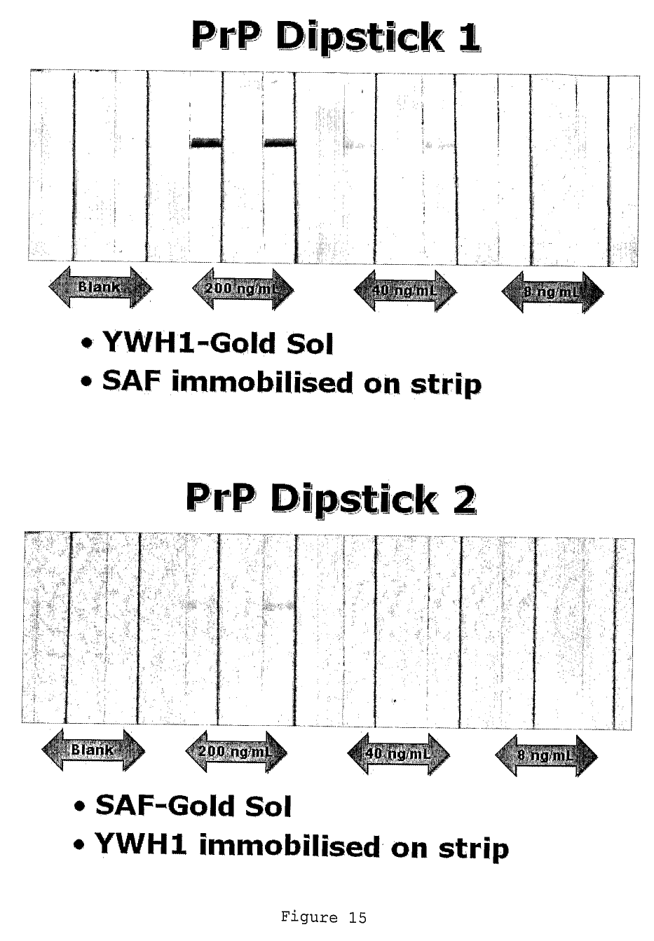

FIG. 15 demonstrates the utility of a PrP specific dipstick in two different configurations.

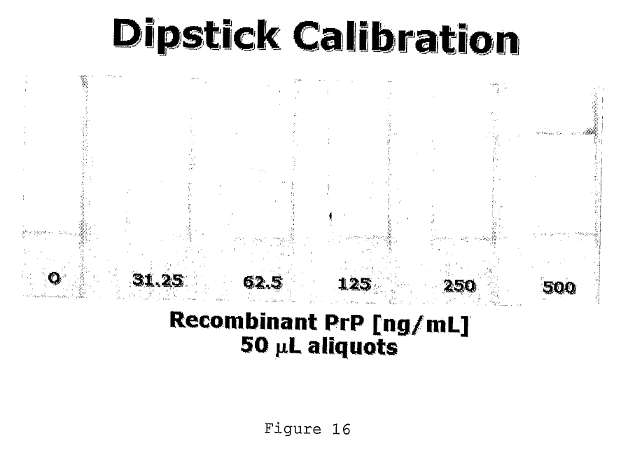

FIG. 16 shows a calibration curve of recombinant PrP using PrP-specific dipstick detection

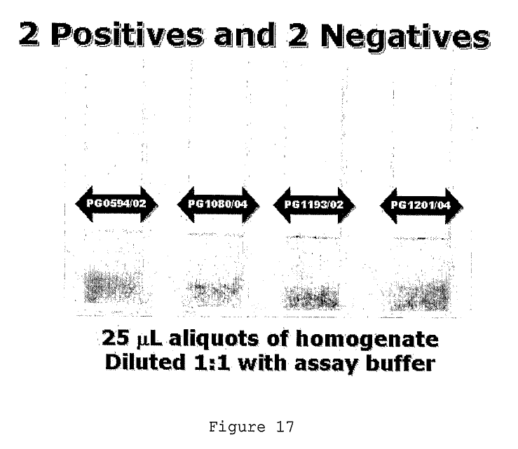

FIG. 17 shows the measurement of PrP in two scrapie-positive and scrapie negative homogenates using PrP-specific dipstick detection

Table 1 shows measurement of disease-associated PrP in histology-confirmed BSE positive and negative bovine caudal medulla

### Materials

### Tissues

Samples of ovine rostral medulla and palatine tonsil were obtained from the Veterinary Laboratories Agency (VLA), New Haw, Addlestone, Surrey KT15 3NB, UK. These included scrapie histology-confirmed positive material and New Zealand (NZ) negative tissue from sheep flocks that had never been exposed to the disease.

In addition, samples of bovine caudal medulla from histology confirmed BSE positives and negatives were made available by the VLA, Whitley Road, Longbenton, Newcastle upon Tyne NE12 9SE, UK. A limited number of human brain homogenates are available from the NIBSC CJD Resource Centre, Blanche Lane, South Mimms, Potters Bar EN6 3QG, UK (Minor, Newham et al. 2004).

### Reagents

The basic components of all buffers and GdHCl (8M; Cat. No. G-9284) were obtained from Sigma-Aldrich Co Ltd., Fancy Road, Poole, Dorset UK. The assay buffer was prepared by dissolving 14.9 g Trizma® preset crystals (pH 7.7; Cat. No. T8068), 18 g sodium chloride (SigmaUltra; Cat. No. S7653); 1 g sodium azide (SigmaUltra; S8032); 16 mg diethylenetriaminepentaacetic acid (DTPA; Cat. No. D6518); 40 mg amaranth (Cat. No. A1016); 0.2 mL Tween 20 (Cat. No. P7949) and 10 g bovine serum albumin (BSA; Cat. No. A3059) in 2 litres of MilliQ water. Mab coating buffer was prepared by dissolving two sachets of PBS powder (pH 7.4; P5368) and 1 g sodium azide (SigmaUltra; S8032) in 2 litres of MilliQ water. Mab blocking buffer was prepared by dissolving two sachets of PBS powder (pH 7.4; P5368), 1 g sodium azide (SigmaUltra; S8032) and 40 g bovine serum-albumin (BSA; Cat. No. A3059) in 2 litres of MilliQ water. All buffers were stored at 4° C. until required.

DELFIA® wash concentrate was prepared by dissolving 72.9 g Trizma® preset crystals (pH 7.7; Cat. No. T8068), 9 g sodium chloride (SigmaUltra; Cat. No. S7653); 1 g sodium azide (SigmaUltra; S8032) and 5 mL Tween 20 (Cat. No. P7949) in 1 litre of MilliQ water. This solution was stored at room temperature. Wash solution was prepared by diluting the concentrate 25-fold in de-ionised water.

DELFIA® enhancement solution (Cat. No. 1244-105) and labelling reagent (Cat. No. 1244-302) were obtained from PerkinElmer UK Ltd, Chalfont Road, Seer Green, Beaconsfield, Buckinghamshire HP9 2FX, UK.

### DELFIA® Equipment

The following equipment obtainable from PerkinElmer is required to perform the DELFIA®: (i) Time-resolved fluorometer plus printer and computer e.g. Victor II (Cat. No. 1420-012 or 1420-016); (ii) Automatic washer e.g. Platewash (Cat. No. 1296-026/R); (iii) Automatic shaker e.g. Plateshake (Cat. No. 1296-003/R); and (iv) Optional dispenser for the Enhancement Solution (Plate Dispense; Cat. No. 1296-041/R).

NUNC Maxisorp® microtitre plates (Cat. No. DIS-971-070U) and plate sealer (Cat. No. DIS-984-505J) were obtained from Fisher Scientific UK Ltd., Bishop Meadow Road, Loughborough, Leicestershire LE11 5RG, UK.

### Tissue Preparation Equipment

A FastPrep® tissue disrupter, grinding tubes containing sucrose buffer and additional polypropylene beads were obtained from Bio-Rad Laboratories Ltd., Bio-Rad House, Maylands Avenue, Hemel Hempstead, Hertfordshire HP2 7TD, UK. Impact resistant 2 mL screw capped grinding tubes containing 0.2 mm ceramic beads (QBIOgene Lysing Matrix D; Cat. No. MBR-247-110Y), 2 mL Safe-Lock® Eppendorf tubes (Cat. No. TUL-150-290U) and a Heraeus Biofuge® Pico micro-centrifuge with 24 place rotor (Cat. No. CFH-230-010H) were obtained from Fisher Scientific UK Ltd. Alternatively, an ETDS VIII (The Design Village, Lowlands Estate, Braye Road, Vale, Guernsey GY3 5XH, Channel Islands) was used to prepare homogenates in GdHCl with and without detergent.

### Immunoassay Standard

Recombinant ovine ARR and VRQ PrP were obtained from the TSE Resource Centre, Institute for Animal Health, Compton, Newbury, Berkshire RG20 7NN, UK. Bovine and Murine PrP were obtained from Biogenesis, Technology Road, Poole, Dorset BH17 7DA, UK. Stock solutions were prepared in 8M GdHCl at a concentration of 20 μg/mL and stored at 4° C. Immediately prior to assay, the stock solution was diluted in assay buffer containing 0.4M-GdHCl to give six working standards, namely, 0, 0.32, 1.6, 8, 40 and 200 ng/mL recombinant ovine PrP.

### Detecting Antibody

Purified BSA-free Mab SAF32 recognising the whole octapeptide repeat region of PrP^{c} spanning residues 51 to 98 (Monnet, Marthiens et al. 2003) was obtained from IDS Ltd, Boldon Business Park, Boldon, Tyne & Wear NE35 9PD, UK. The antibody was labelled with europium according to a method described previously (Barnard and Sy 2003).

### Methods

### Production of Mab YWH1

A series of chimeric peptides were synthesised based on amino acid sequences derived from bovine PrP. One of these involved the very N-terminus of the mature protein (amino acids 25-36) together with one octapeptide repeat (amino acids 62-69). The N-terminal sequence of bovine PrP is illustrated in FIG. 1. The structure of the chimeric peptide immunogen is shown in FIG. 2. Hybridomas were prepared in BALB/c mice according to procedures previously described (Price, Cuthbertson et al. 1990).

### The Production of YWH2 and YWH3 Antibodies Using Phage Display Technology

YWH2 and YWH3 have been identified using proprietary HUCAL® phage-display technology. Briefly, the human N-terminal chimeric peptide antigen (FIG. 3) was screened against the HuCAL® library following immobilisation in 384-well microplates. In the HUCAL® libraries, the structural diversity of the human antibody repertoire is represented by seven heavy chain and seven light chain variable region genes, giving rise to 49 frameworks in the master library. Highly variable genetic cassettes are then superimposed on these frameworks to mimic the entire human antibody repertoire. More than 10 billion functional human antibody specificities in Fab format are already prefabricated and available in the phage libraries.

Proprietary technology (CysDisplay™) provided a simple elution of high-affinity binders and the candidates were screened in a robust 384-well ELISA. The two positive clones (YWH2 and YWH3) were expressed in bacteria and affinity purified. The basic antibody format is a monovalent Fab fragment, but in the case of YWH2 and YWH3, these were converted into bivalent mini-antibodies with a C-terminal Myc-tag (EQKLISEEDL).

### Preparation of the YWH-Coated Plates

Purified YWH antibodies (types 1, 2 and 3) were diluted in PBS coating buffer to a concentration of 5 μg/mL. Two hundred (200) μL of coating solution (1 μg IgG per well) were added to each well of NUNC Maxisorp®microtitre plates using a 12-channel digital pipette. The plates were sealed and stored overnight at 4° C. Subsequently, the plates were washed once with wash solution and tapped dry on absorbent paper. Two hundred (200) μL of blocking buffer were added to each well, the plates re-sealed and stored at 4° C. until use.

### Homogenisation and Extraction of Tissue

Approximately 300 mg brain or lymphoid tissue were carefully cut, weighed and transferred to Bio-Rad grinding tubes containing sucrose buffer, 0.2 mm ceramic beads and one larger polypropylene bead. In addition, approximately 250 mg brain or lymphoid tissue were carefully cut, weighed and transferred to QBIOgene grinding tubes (i.e. without buffer) containing 0.2 mm ceramic beads and one larger polypropylene bead. Subsequently, 800 μL 8M-GdHCl was added to the QBIOgene grinding tubes. All grinding tubes were stoppered, cooled on ice, transferred to the FastPrep® tissue disrupter and processed twice for 45 seconds at maximum speed according to the manufacturer's instructions.

Fifty (50) μL of the sucrose homogenates were carefully pipetted into 2 mL Eppendorf Safe-Lock® tubes and 50 μL 8MGdHCl added. The tubes were stoppered and vortexed. Subsequently, 900 μL of assay buffer were added, the tubes stoppered, vortexed and then spun in a microfuge at 13,000 rpm for 5 min.

Alternatively, tissue was cut, accurately weighed and transferred to the ETDS VIII homogeniser tube. An equivalent volume of 8M GdHCl (with or without detergent) was added. The tube was assembled and the tissue homogenised for 20 seconds (brain) or 40 seconds (lymphoid tissue) using the ETDS VIII homogeniser. Subsequently, the homogenates were diluted by the addition of assay buffer (through the central tube of the disposable device). The diluted homogenates were mixed using the EDTS VIII homogeniser for a further 10 seconds and used without additional treatment.

### Immunoassay

YWH-coated microtitre plates were washed twice with wash solution and tapped dry on absorbent paper. Two hundred (200) μL of each standard or sample were added in duplicate to appropriate wells of the coated plate. The plates were sealed and incubated on the shaker for 2 hours at 18° C. Subsequently, the plates were washed three times with wash solution, tapped dry on absorbent paper, and 200 μL of europium-labelled anti-PrP detecting antibody (typically SAF32; diluted at 1:2000 v/v in assay buffer) added to each well. The plates were incubated on the shaker at 18° C. for a further 60 mins. The plates were then washed six times, tapped dry, turned around 180°, and re-washed a further six times. The plates were again tapped dry on absorbent paper.

Two hundred (200) μL of enhancement solution were added, the plates shaken for 5 mins at 18° C. and the fluorescence measured in the time-resolved fluorometer. The concentrations of PrP were determined using the proprietary PerkinElmer data reduction package Multicalc®.

### Results

### Calibration Curves of YWH1, YWH2 and YWH3

Calibration curves of recombinant ovine and human PrP for the three YWH antibodies are shown in FIG. 4 (Linear Y-axis) and FIG. 5 (Logarithmic Y-axis). The minimum detectable concentration of PrP, as calculated by Multicalc®, was typically less than 20 pg/mL (equivalent to 4 pg PrP per well). FIG. 4 shows that the three antibodies have slightly different specificities. YWH1 is more specific for ovine and bovine but only has limited cross-reactivity with human PrP. On the other hand, YWH2 recognizes ovine and human PrP equally. YWH3 has slightly higher cross-reactivity with human but is somewhat compromised by the fact that it gives high backgrounds with recombinant protein.

### Cross Reactions of YWH1, YWH2 and YWH3

A series of overlapping peptides were prepared to map the epitope for the YWH series of antibodies. To establish competitive blocking assays, each of these peptides was diluted in assay buffer containing 2 ng/mL recombinant ovine PrP to give a range of peptide concentrations (i.e. 0, 100, 1000 and 10,000 ng/mL). Two hundred (200) μL of each dilution were added in duplicate to appropriate wells of the coated plate. The plates were incubated on the shaker for 2 hours at 18° C. Subsequently, the plates were washed three times with wash solution, tapped dry on absorbent paper, and 200 μL of europium-labelled anti-PrP detecting antibody (typically SAF32; diluted at 1:1000 v/v in assay buffer) added to each well. The plates were incubated on the shaker at 18° C. for a further 60 min. The plates were then washed six times, tapped dry, turned around 180°, and re-washed a further six times. The plates were again tapped dry on absorbent paper. Two hundred (200) μL of enhancement solution were added, the plates shaken for 5 min at 18° C. and the fluorescence measured in the time-resolved fluorometer. The results are shown in FIG. 6.

These results show that YWH1 sees an epitope at the C-terminal end of the N-terminal peptide (i.e. PGGGWNT). In particular, a triple glycine motif is required for strong interaction and this explains the ovine/bovine specificity of this reagent and the somewhat limited cross-reaction with human PrP which only has a double glycine motif at the N-terminus. The epitope for YWH2 is in the basic region of the extreme N-terminus (i.e. KKRPKPG). This explains why this reagent recognises ovine as well as human PrP as this epitope is conserved between species. The cross reactions with YWH3 are more complex and this might explain the high backgrounds obtained using this reagent.

### Measurement of Disease-Associated PrP in Histology-Confirmed BSE Positive and Negative Bovine Brain Tissue

The concentrations of disease-associated PrP were determined in 400 samples of bovine brain tissue taken caudal to the obex (200 histology-confirmed positives and 200 histology-confirmed negatives) using Mab YWH1 and the results are shown in Table 1. These results show that YWH1 can clearly distinguish between BSE positives and negatives (p<0.001). There is, however, a wide standard deviation in the positive samples and this reflects the differing levels of PrP^{d} in these tissues.

### Measurement of Disease-Associated PrP Using YWH Series Antibodies in Scrapie Positive and Negative Tissues Following Homogenisation in Sucrose or GdHCl

The concentrations of disease-associated PrP were determined in samples of ovine tissue, following homogenisation in either sucrose or GdHCl, using YWH1, YWH2 and YWH3. The results shown in FIG. 7 show that YWH3 is relatively poor at recognizing tissue-derived PrP. This may be due to non-specific binding problems. On the other hand, YWH1 and YWH2 can clearly identify tissues taken from scrapie-positive animals following homogenisation with either sucrose buffer or 8M GdHCl. Accordingly, in all subsequent experiments, only YWH1 and YWH2 were used.

### Measurement of Disease-Associated PrP in Histology-Confirmed Scrapie Positive and Negative Ovine Palatine Tonsil

The concentrations of disease-associated PrP were determined in samples of ovine palatine tonsillar tissue using YWH1 and YWH2 and the results are shown in FIG. 8. These assays were done blind prior to the histochemical confirmation of scrapie diagnosis, which was performed by the VLA on obex brain tissue. The lymphoid tissue assay was able to detect the scrapie positives with 100% concordance.

### Measurement of Disease-Associated PrP in Histology-Confirmed Scrapie Positive and Negative Ovine Brain Tissue

Similarly, concentrations of disease-associated PrP were determined in equivalent samples of ovine brain tissue taken rostral to the obex using YWH1 and YWH2 and the results are shown in FIG. 9.

Measurement of Disease-Associated PrP in Normal and vCJD Human Tissue

The concentrations of disease-associated PrP were determined with YWH1 and YWH2 in duplicate samples of homogenate prepared from vCJD, sporadic CJD, normal brain and normal tonsil tissue. The results are shown in FIG. 10. We have not been able to test vCJD tonsil tissue but in order to demonstrate that PrP^{d} can be detected in a tonsil matrix, 3 human tonsil homogenates were spiked with vCJD brain homogenate to produce mixtures containing 100%, 37.5%, 12.5%, 2.8%, 1.4%, 0.07% and 0% vCJD brain tissue. The concentrations of disease-associated PrP were determined with YWH2 and the results are shown in FIG. 11.

### Measurement of Disease-Associated PrP in Normal and Scrapie Positive Sheep Brain Tissue by GdHCl in the Presence and Absence of Detergent

The concentrations of disease-associated PrP were determined with YWH1 and YWH2 in duplicate samples of homogenate (2 negatives) prepared in GdHCl containing variable amounts of detergent (Tween 20 or Triton X-100). The results are shown in FIG. 12. The results indicate that the presence of detergent may increase the efficiency of both PrP^{c} and PrP^{d} extraction.

### Measurement of Disease-Associated PrP in Normal and Scrapie Positive Sheep Brain Tissue by GdHCl Using Alternative Antibodies.

The concentrations of disease-associated PrP were determined with alternative combinations of Mabs including (i) YWH1, YWH2 and SAF32; (ii) 11G5 (epitope specificity 115-130), 8B4 (epitope specificity 35-45), 4H2 (epitope specificity 145-155); (iii) P4 (epitope specificity 89-105) obtained from R-Biopharm Rhone Ltd., Scotland Science Park, Kelvin Campus, Glasgow G20 OSP; and (iv) FH11 (epitope specificity 63-95) obtained from the TSE Resource Centre, Institute for Animal Health, Compton, Berkshire, UK) using duplicate samples of homogenates (5 negatives and 1 positive) prepared in 8M GdHCl or 8MGdHCl containing 5% Triton X-100. The results are shown in FIG. 13A (no detergent) and FIG. 13B (with detergent). The results indicate that operational specificity can be achieved with non-YWH antibodies (N-terminal and C-terminal) although the ability to discriminate between normal and disease is significantly diminished.

### Effect of Homogenates on a Lateral Flow Device

Negative sheep brain homogenate was prepared in 8M GdHCl and diluted as described. Human chorionic gonadotrophin (hCG) was spiked into an aliquot of this diluted homogenate to a concentration of 100 miU/mL. Spiked and non-spiked homogenate was applied to two different types of pregnancy test (Abbott Laboratories and Boots own brand). The results are shown in FIG. 14. The results indicate that the diluted homogenates do not impair the flow of a dipstick.

### Measurement of Recombinant PrP Using Lateral Flow Detection

PrP-specific dipstick devices were produced. Two configurations were developed. In the first configuration, YWH1 was conjugated to gold sol particles as previously described (Martin JM et al. J Immunoassay 1990 11: 31-47) and SAF Mabs were immobilised onto nitrocellulose strips according to known procedures (e.g. U.S. Pat. No. 5,712,172). In the second configuration, SAF Mabs were conjugated to the gold sol particles (as above) and YWH1 was immobilised onto nitrocellulose strips (as above). The results of the two configurations with recombinant PrP at three dilutions (equivalent to 200 ng/mL, 40 ng/mL and 8 ng/mL) together with an appropriate blank are shown in FIG. 15. Configuration 1 was shown to be more sensitive and a full calibration curve using this format is shown in FIG. 16.

### Measurement of Disease-Associated PrP in Normal and Scrapie Positive Sheep Brain Tissue by GdHCl Using Lateral Flow Detection

The presence of disease-associated PrP in diluted homogenates was detected with YWH1 using a dipstick detection device (configuration 1). The results of two scrapie confirmed positives and scrapie confirmed negatives are shown in FIG. 17.

The production and use of unique anti-PrP^{d} antibodies are described herein. These antibodies include a reagent that has been produced by conventional Mab production technology (YWH1) and two reagents (YWH2 and YWH3) that have been identified using phage display technology. All three antibodies recognize an epitope at the extreme N-terminus of PrP. One of the significant characteristics of the antibodies is their relative high affinity for a linear epitope. This epitope is present in recombinant PrP and the specificity and sensitivity are illustrated by the calibration curves for ovine and human PrP shown in FIG. 4 (Linear Y-axis) and FIG. 5 (Logarithmic Y-axis).

The three antibodies have slightly different specificities. YWH1 is more specific for ovine and bovine but only has limited cross-reactivity with human PrP. On the other hand, YWH2 recognizes ovine and human PrP equally. YWH3 has slightly higher cross-reactivity with human but is somewhat compromised by the fact that it gives high backgrounds with recombinant protein. All of these findings are supported by the cross-reactivity data shown in FIG. 6. From these studies it has been possible to establish that YWH1 sees an epitope at the C-terminal end of the N-terminal peptide (i.e. PGGGWNT). On the other hand, the epitope for YWH2 is in the basic region of the extreme N-terminus (i.e. KKRPKPG). This explains why this reagent recognises ovine as well as human PrP as this epitope is conserved between species.

The robust detection methods described herein are exquisitely sensitive and the limit of detection is routinely less than 5 pg PrP per well. This is sufficiently sensitive to detect aggregated PrP in tonsil and other lymphoid tissue (see FIGS. 8, 10 and 11). Accordingly, the methods may be useful in tracking the development of disease from its earliest stages in blood or primary lymphoid tissue to the terminal stages of the disease when the aggregated protein is present in relatively high levels in the CNS (see FIG. 9 and Table 1).

Furthermore, the specificity of the Mab obviates the need for differential proteolysis to discriminate between PrP^{c} and PrP^{d}. The simple one-step extraction procedure, therefore, lends itself to full-automation, which is significant when large scale testing is required. In particular, we have demonstrated that this approach lends itself to the development of specific and rapid lateral flow detection.

### REFERENCES

Atarashi, R. et al. (2003). J Biol Chem 278(31): 28944-9.
Barnard, G. et al. (2000). Luminescence 15(6): 357-62.
Barnard, G. and M-S. Sy (2003). Prions and Mad Cow Disease. I.
Krull. New York, Marcel Dekker: 277-315.
Biffiger, K. (2002) J Virol Methods 101 (1-2): 79-84.
Buschmann A. et al. (2004) J Virol Methods 117(1): 27-36.
Caughey, B. et al. (1999) Methods Enzymol 309: 122-33.
Ciechanover, A. and P. Brundin (2003) Neuron 40(2): 427-46.
Cooley, W. A. et al. (2001). J Comp Pathol 125(1): 64-70.
Cicortas Gunnarsson L. et al. (2004) Protein Eng Des Sel 17:213-21
Curin Serbec, V. et al. (2003) J Biol Chem.
Eaglestone S. et al (2000) Proc Nat Acad Sci USA 97(1): 240-4.
Epple, G. et al. (2004) Thromb Haemost 91(3): 465-72.
Gorochov, G. and J. P. Deslys (2004) Nat Med 10(1): 11; author reply 11-2.
Grassi, J. et al. (2001). Vet Rec 149(19): 577-82.
Hamir, A. N. et al. (2001). J Vet Diagn Invest 13(2): 152-4.
Heggebo, R. et al. (2003). J Comp Pathol 128 (2-3): 172-81.
Hooper, N. M. (2003) Trends Biotechnol 21(4): 144-5.
Kang, S. C. et al. (2004) J Pathol 203(1): 603-8.
Kang, S. C. et al. (2003) J Pathol 199(4): 534-41.
Kascsak, R. et al. (1997) Immunol Invest 26(1-2): 259-68.
Kocisko, D. A. et al. (1996) Biochemistry 35(41): 13434-42.
Koide A, Bailey C W, Huang X, Koide S. (1998) J Mol Biol 11:1141-51.
Korth, C. et al. (1997) Nature 390(6655): 74-7.
Kubler, E. et al. (2003). Br Med Bull 66: 267-79.
Lawson, V. A. et al. (2004) J Biol Chem 279(14): 13689-95.
Lee, K. S. et al. (2001) J Neurochem 79(1): 79-87.
Lucero, H. A. and P. W. Robbins (2004) Arch Biochem Biophys 426(2): 208-24.
Lundberg, P. et al. (2002) Bioch Biophys Res Comm 299(1):85-90.
Ma, J. and S. Lindquist (2002) Science 298(5599): 1785-8.
Ma, J., R. Wollmann, et al. (2002). Science 298(5599): 1781-5.
Madec, J. Y. et al. (2000) Vet Rec 146(3): 74-6.
Madec, J. Y. et al. (1998) J Virol Methods 75(2): 169-77.
Martin J. M. et al. (1990) J Immunoassay 11: 31-47
Minor, P. et al. (2004) J Gen Virol 85 (Pt 6): 1777-84.
Mishra, R. S. et al. (2003). J Alzheimers Dis 5(1): 15-23.
Moffett, S. et al. (2000) J Biol Chem 275(3): 2191-8.
Monnet, C. et al. (2003) European J Neuroscience 18(3): 542-9.
Moynagh, J. and H. Schimmel (1999) Nature 400(6740): 105.
Oesch, B. et al. (2000) Arch Virol Suppl (16): 189-95.
Paramithiotis, E. et al. (2003) Nat Med 9(7): 893-9.
Price, K. M. et al. (1990) Dev Biol Stand 71: 23-31.
Prusiner, S. B. (1998). Brain Pathol 8(3): 499-513.
Rothe, A. et al. (2006) FASEB J 20:1599-610.
Safar, J. et al. (1993). J Biol Chem 268(27): 20276-84.
Safar, J. et al. (1998). Nat Med 4(10): 1157-65.
Safar, J. G. et al. (2002) Nat Biotechnol 20(11): 1147-50.
Sakudo, A. et al. (2005) Bioch Biophys Res Comm 326 (3:600-606.
Sarnataro, D. et al. (2004) Mol Biol Cell 15(9): 4031-42.
Schaller, O. et al. (1999). Acta Neuropathol (Berl) 98 (5): 437-43.
Stack, M. J. et al. (2002 Acta Neuropathol (Berl) 104(3):279-86.
Tenzer, S. et al. (2004). J Immunol 172(2): 1083-91.
van Keulen, L. J. et al. (2002 Apmis 110(1): 23-32.
van't H of, W. and M. D. Resh (2000) Meth Enzymol 327: 317-30.
Walmsley, A. R. et al. (2003) J Biol Chem 278(39): 37241-8.
Wang, X., F. Wang, et al. (2005). J Biol Chem 280(1): 317-25.
Wong, E. et al. (2004) Biochem J 380 (Pt 1): 273-82.
Yedidia, Y. et al. (2001) Embo J 20(19): 5383-91.
Zahn, R. (2003) J Mol Biol 334(3): 477-88.
Zanusso, G. et al. (1999) J Biol Chem 274(33): 23396-404.
Zeng, F. et al. (2003) J Neurochem 84(3): 480-90.

TABLE 1

| Negatives | | | Positives | | |
|---|---|---|---|---|---|
| MEAN | 0.046 | ng/mL | MEAN | 14.22 | ng/mL |
| ST. DEV. | 0.075 | ng/mL | ST. DEV. | 18.07 | ng/mL |
| Range | 0.01 to 0.253 | ng/mL | Range | 0.928 to 80.25 | ng/mL |

| | | | |
|---|---|---|---|
| N | 200 | N | 200 |

Sequences

| | |
|---|---|
| SEQ ID | MANLGCWMLVLFVATWSDLGLC-KKRPKPGGWNTGGSR |
| NO:1 | |
| SEQ ID | MVKSHIGSWILVLFVAMWSDVGLC-KKRPKPGGGWNTGGSR |
| NO:2 | |
| SEQ ID | MANLGYWLLALFVTMWTDVGLC-KKRPKPGGWNTGGSR |
| NO:3 | |

Signal Peptide Underlined

1.-78. (canceled)

79. A method of detecting the presence of a disease-form prion protein (PrP^{d}) in a sample comprising:
contacting said sample with a first specific binding member which specifically binds to an amino acid sequence selected from the group consisting of:
(SEQ ID NO: 1)
MANLGCWMLVLFVATWSDLGLCKKRPKPGGWNTGGSR,
(SEQ ID NO: 2)
MVKSHIGSWILVLFVAMWSDVGLCKKRPKPGGGWNTGGSR,
and
(SEQ ID NO: 3)
MANLGYWLLALFVTMWTDVGLCKKPKPGGWNTGGSR;
and
determining the binding of said first specific binding member to said sample, wherein binding of said first specific binding member to said sample is indicative that the sample contains said disease-form prion protein.

80. A method according to claim 79 wherein the first specific binding member binds specifically to an amino acid sequence selected from the group consisting of KKRPKPGGGWNT (SEQ ID NO: 4) and KKRPKPGGWNT (SEQ ID NO: 5).

81. A method according to claim 80 wherein the first specific binding member binds specifically to the amino acid sequence KKRPKPG (SEQ ID NO: 6).

82. A method according to claim 80 wherein the first specific binding member binds specifically to the amino acid sequence PGGGWNT (SEQ ID NO: 7).

83. A method according to claim 79 wherein the first specific binding member binds preferentially to disease-form prion protein (PrP^{d}) relative to cellular prion protein (PrP^{c}).

84. A method according to claim 83 wherein the first specific binding member binds to PrP^{d} and shows substantially no binding to PrP^{c}.

85. A method according to claim 79 wherein the first specific binding member is an antibody molecule.

86. A method according to claim 85 wherein the first specific binding member is produced by the hybridoma with the ECCAC deposit number 05091301.

87. A method according to claim 85 wherein the first specific binding member competes for binding to PrP^{d} with the antibody produced by the hybridoma with the ECCAC deposit number 05091301.

88. A method according to claim 85 wherein the first specific binding member comprises the V_{H}CDR1, V_{H}CDR2 and V_{H}CDR3 sequences and V_{L}CDR1, V_{L}CDR2 and V_{L}CDR3 sequences of the antibody produced by the hybridoma with the ECCAC deposit number 05091301.

89. A method according to claim 79 wherein the first specific binding member comprises a detectable label.

90. A method according to claim 79 wherein the binding of the first specific binding member is determined using a second specific binding member.

91. A method according to claim 90 wherein said second specific binding member binds to said first specific binding member.

92. A method according to claim 91 wherein said second specific binding member comprises a detectable label.

93. A method of detecting a disease-form prion protein in a sample comprising:
contacting said sample with a first specific binding member; and
determining binding of said first specific binding member to a molecule in said sample using a second specific binding member, wherein the presence of said binding is indicative of the presence of said disease-form prion protein; and wherein one of said first or second specific binding member is a specific binding member which specifically binds to an amino acid sequence selected from the group consisting of:
(SEQ ID NO: 1)
MANLGCWMLVLFVATWSDLGLCKKRPKPGGWNTGGSR,
(SEQ ID NO: 2)
MVKSHIGSWILVLFVAMWSDVGLCKKRPKPGGGWNTGGSR,
and
(SEQ ID NO: 3)
MANLGYWLLALFVTMWTDVGLCKKRPKPGGWNTGGSR;
and
the other of said first or second specific binding member binds to both PrP⁰ and PrP^{d}.

94. A method according to claim 93 wherein one of said first and said second specific binding member is immobilised.

95. A method according to claim 94 wherein the non-immobilised specific binding member of said first and second specific binding members comprises a detectable label.

96. A method according to claim 95 wherein the binding of said first specific binding member to said molecule in said sample is determined by detecting the presence of the detectable label.

97. A method according to claim 79 further comprising solubilising the protein in said sample.

98. A method according to claim 97 wherein the protein is solubilised by treating the sample with a solubilising agent.

99. A method according to claim 98 wherein the solubilising agent is a chaotropic agent.

100. A method according to claim 79 wherein the sample is obtained from a non-biological surface.

101. A method according to claim 79 wherein the sample is obtained from an individual.

102. A method according to claim 101 wherein the presence of said disease-form prion protein is indicative that the individual has a transmissible spongiform encephalopathy (TSE).

103. A method according to claim 101 wherein the sample is a tissue or a biological fluid of said individual.

104. A method according to claim 103 wherein the sample is disrupted in the presence of a solubilising agent.

105. A method according to claim 79 wherein the sample is not protease treated.

106. A kit for detecting the presence of PrP^{d} in a sample comprising:
one or more first specific binding members which specifically binds to an amino acid sequence selected from the group consisting of:
(SEQ ID NO: 1)
MANLGCWMLVLFVATWSDLGLCKKRPKPGGWNTGGSR,
(SEQ ID NO: 2)
MVKSHJGSWILVLFVAMWSDVGLCKKRPKPGGGWNTGGSR,
and
(SEQ ID NO: 3)
MANLGYWLLALFVTMWTDVGLCKKRPKPGGWNTGGSR;
and
a detection reagent for determining binding of said binding member to the sample.

107. A kit according to claim 106 comprising apparatus for handling and/or storing tissue obtained from the individual.

108. A kit according to claim 106 further comprising reagents for homogenising and/or extracting said sample from said tissue.

109. A kit according to claim 106 further comprising a solubilising agent.

110. A kit according to claim 106 wherein the detection reagent is a second specific binding member that binds to both PrP^{c} and PrP^{d}.

111. A kit according to claim 110 wherein the one or more first specific binding members or the second specific binding member is immobilised.

112. A kit according to claim 111 wherein the one or more first specific binding members or the second specific binding member is immobilised in a lateral flow device.

113. A kit according to claim 110 wherein the one or more first specific binding members or the second specific binding member is labeled with a detectable label.

114. A method of screening for a compound useful in the detection of infectious prion proteins comprising:
contacting a test compound with a peptide consisting of a sequence selected from the group consisting of SEQ ID NOS: 1-3 and fragments thereof; and
determining the binding of the compound to the peptide, wherein binding of said compound to the peptide is indicative that the compound is useful in the detection of infectious prion proteins.


SEQUENCE LISTING (15 sequences)

SEQ ID NO: 1 - protein, 37 aa, Homo sapiens
MANLGCWMLVLFVATWSDLGLCKKRPKPGGWNTGGSR

SEQ ID NO: 2 - protein, 40 aa, Bos sp.
MVKSHIGSWILVLFVAMWSDVGLCKKRPKPGGGWNTGGSR

SEQ ID NO: 3 - protein, 37 aa, Murine
MANLGYWLLALFVTMWTDVGLCKKRPKPGGWNTGGSR

SEQ ID NO: 4 - protein, 12 aa, Bos sp.
KKRPKPGGGWNT

SEQ ID NO: 5 - protein, 11 aa, Homo sapiens
KKRPKPGGWNT

SEQ ID NO: 6 - protein, 7 aa, Homo sapiens
KKRPKPG

SEQ ID NO: 7 - protein, 7 aa, Bos sp.
PGGGWNT

SEQ ID NO: 8 - protein, 10 aa, Artificial sequence
  Synthetic sequence C-terminal Myc-tag
EQKLISEEDL

SEQ ID NO: 9 - protein, 79 aa, Bos sp.
KKRPKPGGGWNTGGSRYPGQGSPGGNRYPPQGGGGWGQPHGGGWGQPHGGGWGQPHGGGWGQPHGGGWGQPHGGGGWGQ

SEQ ID NO: 10 - protein, 20 aa, Artificial sequence
  Synthetic sequence N-terminal bovine chimeric peptide used for the immunogen for the production of YWH1
KKRPKPGGGWNTQPHGGGWG

SEQ ID NO: 11 - protein, 19 aa, Artificial sequence
  Synthetic sequence N-terminal human chimeric peptide used as the antigen in the panning for YWH2 and YWH3
KKRPKPGGWNTQPHGGGWG

SEQ ID NO: 12 - protein, 7 aa, Bos sp.
KRPKPGG

SEQ ID NO: 13 - protein, 7 aa, Bos sp.
RPKPGGG

SEQ ID NO: 14 - protein, 7 aa, Bos sp.
PKPGGGW

SEQ ID NO: 15 - protein, 7 aa, Bos sp.
KPGGGWN